SEQUENCE LISTING (4 sequences)

SEQ ID NO: 1 - protein, 39 aa, Artificial Sequence
  Synthetic Construct
  AMIDATION 39
HGEGTFTSDLSKQMEEEAVRLFIEWLKNGGPSSGAPPPS

SEQ ID NO: 2 - protein, 44 aa, Artificial Sequence
  Synthetic Construct
  AMIDATION 44
HGEGTFTSDLSKQMEEEAVRLFIEWLKNGGPSSGAPPSKKKKKK

SEQ ID NO: 3 - protein, 31 aa, Artificial Sequence
  Synthetic Construct
  VARIANT 20: Xaa = Lysine modified with C-16 fatty acid (palmitic acid) with glutamic acid spacer
HAEGTFTSDVSSYLEGQAAXEFIAWLVRGRG

SEQ ID NO: 4 - protein, 38 aa, Artificial Sequence
  Synthetic Construct
  AMIDATION 38
GEGTFTSDLSKQMEEEAVRLFIEWLKNGGPSSGAPPPS

(19) United States
(12) Patent Application Publication
(10) Pub. No.: US 2017/0313740 A1
(43) Pub. Date: Nov. 2, 2017

(54) METHODS OF PREPARING PEPTIDES

(71) Applicant: C S Bio Co., Menlo Park, CA (US)

(72) Inventors: Wei WANG, Fremont, CA (US); Jason CHANG, Palo Alto, CA (US); Dario SLAVAZZA, Fremont, CA (US); Heng Wei CHANG, Foster City, CA (US)

(21) Appl. No.: 15/499,793

(22) Filed: Apr. 27, 2017

Related U.S. Application Data
(60) Provisional application No. 62/329,097, filed on Apr. 28, 2016.

(51) Int. Cl. C07K 1/04; C07K 14/00

(52) U.S. Cl. CPC C07K 1/04; C07K 14/001

(57) ABSTRACT

The present invention provides methods of preparing a peptide having an N-terminal histidine, and compositions comprising a plurality of peptides prepared by the methods. The methods disclosed herein reduce racemization of the N-terminal histidine in the peptides during the synthesis process, thereby improving the yield and purity of the peptide compositions. Exemplary peptides that can be manufactured with the methods include Exenatide, Lixisenatide, and Liraglutide.

### CROSS-REFERENCE TO RELATED APPLICATIONS

This application claims priority benefit of U.S. Provisional Patent Application No. 62/329,097, filed on Apr. 28, 2016, the contents of which are hereby incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 725542000100SEQLIST.txt, date recorded: Apr. 27, 2017, size: 3 KB).

### FIELD OF THE INVENTION

The present invention relates to the field of polypeptide synthesis, and particularly, to methods of preparing peptides having an N-terminal histidine.

### BACKGROUND OF THE INVENTION

Exenatide and its analog Lixisenatide are glucagon-like peptide-1 agonists that enhance glucose-dependent insulin secretion by the pancreatic beta-cell, suppress inappropriately elevated glucagon secretion, and slow gastric emptying. Exenatide was approved by the U.S. FDA in 2005 for treating type II diabetes in patients whose condition was not well-controlled on other oral medication. Commercially available drug products of Exenatide are administered by subcutaneous injection twice per day (BYETTA®) or weekly (BYDUREON®). Lixisenatide (LYXUMIA®) was approved by the European Medicines Agency in 2013 as a once-daily injection drug for treating adults with type II diabetes in combination with oral glucose-lowering medicinal products and/or basal insulin.

Exenatide and Lixisenatide can be synthesized by solid-phase peptide synthesis (SPPS) methods. See, for example, U.S. Pat. No. 6,924,264, U.S. Pat. No. 7,157,555, U.S. Pat. No. 6,902,744, U.S. Pat. No. 6,528,486, CN101357938A, WO2014067084A1, CN102558338B, WO2005058954A1, WO2001004156A1, and CN104211801A. Several types of impurities with D-form amino acids, including D-His, can be found at significant levels in the final Exenatide or Lixisenatide products manufactured using current SPPS methods. Typical D-His-Exenatide impurity level measured from several lots of Exenatide Acetate synthesized with a standard Fmoc SPPS method was found to be between about 1.8% and about 2.9%, well above the acceptable GMP criterion of less than 1.0%. The D-His impurities seriously compromise the quality and safety of Exenatide and Lixisenatide products for medical use.

Standard peptide purification methods using a reverse-phase C18 column on HPLC do not effectively separate the D-His impurity from the desired L-His product. Although Strong Cation-Exchange (SCX) HPLC can be used to resolve the D-His impurity peak from the L-His product peak, such purification method is not practical for large scale manufacturing. Thus, there exists a need for an improved method of synthesizing peptides with an N-terminal enantiomerically pure histidine, such as Exenatide and Lixisenatide, which provides high yield and low D-His impurities, and is amenable for industrial scale production.

The disclosures of all publications, patents, patent applications and published patent applications referred to herein are hereby incorporated herein by reference in their entirety.

### BRIEF SUMMARY OF THE INVENTION

The present invention provides methods for preparing peptides with an N-terminal histidine, compositions (such as pharmaceutical compositions) prepared using the methods, and methods of treating a disease or condition (such as diabetes) using the compositions.

One aspect of the present application provides a method of preparing a peptide having an N-terminal histidine, comprising: (a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the peptide having the N-terminal histidine. In some embodiments, the method further comprises synthesizing the resin-bound peptide intermediate on a resin using Fmoc-protected amino acids according to the sequence of the peptide having the N-terminal histidine. In some embodiments, the side chain of the N-terminal histidine of the resin-bound peptide intermediate is protected by a group selected from trityl (Trt), 4-methyltrityl (Mtt), and p-methoxytrityl (Mmt).

In some embodiments according to any one of the methods of preparation described above, the acidic cleavage solution comprises trifluoroacetic acid (TFA). In some embodiments, the acid cleavage solution comprises water. In some embodiments, the acid cleavage solution comprises a scavenger, such as EDT and/or TIS. In some embodiments, the acidic cleavage solution comprises TFA, TIS, EDT, and H₂O, at a volume ratio of about 94:2:2:2. In some embodiments, the resin-bound peptide intermediate is contacted with the acidic cleavage solution for about 2 hours to about 5 hours.

In some embodiments according to any one of the methods of preparation described above, the basic deblock solution comprises piperidine. In some embodiments, the basic deblock solution comprises acetonitrile (ACN). In some embodiments, the basic deblock solution comprises water. In some embodiments, the concentration of the piperidine in the deblock solution is about 10% to about 25% (such as about 15% to about 25%, or about 10% to about 20%) by volume. In some embodiments, the resin-free peptide intermediate is contacted with the basic deblock solution for about 15 minutes to about 30 minutes.

In some embodiments according to any one of the methods of preparation described above, step (a) provides a crude mixture of the resin-free peptide intermediate, and the crude mixture is contacted with the basic deblock solution in step (b).

In some embodiments according to any one of the methods of preparation described above, the method further comprises contacting the reaction mixture comprising the peptide having the N-terminal histidine with an acidic neutralization solution after step (b). In some embodiments, the acidic neutralization solution comprises TFA (such as 50% TFA in water).

In some embodiments according to any one of the methods of preparation described above, the method further comprises purifying the peptide having the N-terminal histidine. In some embodiments, the peptide having the N-terminal histidine is purified using chromatography, e.g., using a reverse-phase column. In some embodiments, the peptide having the N-terminal histidine is purified using a C18 column. In some embodiments, the mobile phase of the reverse-phase column comprises TFA, ACN and water.

In some embodiments according to any one of the methods of preparation described above, the N-terminal histidine is an L-histidine. In some embodiments, the peptide is Exenatide, such as the peptide having the amino acid sequence of SEQ ID NO:1. In some embodiments, the peptide is Lixisenatide, such as the peptide having the amino acid sequence of SEQ ID NO:2. In some embodiments, the peptide is Liraglutide, such as the peptide having the amino acid sequence of SEQ ID NO:3.

One aspect of the present application provides a composition comprising a plurality of peptides having an N-terminal histidine prepared by any one of the methods of preparation described above, wherein the percentage of peptides having an N-terminal D-histidine in the composition is less than about 1% (such as less than about any of 0.75%, 0.5%, or 0.3%). In some embodiments, the composition comprises Exenatide. In some embodiments, the composition comprises Lixisenatide. In some embodiments, the composition comprises Liraglutide.

One aspect of the present application provides a composition (such as a pharmaceutical composition) comprising a plurality of peptides having an N-terminal histidine, wherein the percentage of peptides having an N-terminal D-histidine in the composition is less than about 1% (such as less than about any of 0.75%, 0.5%, or 0.3%). In some embodiments, the composition comprises Exenatide. In some embodiments, the composition comprises Lixisenatide. In some embodiments, the composition comprises Liraglutide.

Further provided by one aspect of the present application is a method of treating a disease or condition in an individual in need thereof, comprising administering to the individual an effective amount of any one of the composition of described above. In some embodiments, the disease is diabetes, such as type II diabetes.

One aspect of the present application provides a composition comprising a plurality of resin-free peptide intermediates, wherein each resin-free peptide intermediate comprises an Fmoc-protected N-terminal histidine and unprotected amino acid side chains.

Another aspect of the present application provides a commercial batch of any one of the compositions described above. In some embodiments, the size of the commercial batch is about 1 gram to about 10 Kg.

Further provided are kits, and articles of manufacture comprising any one of the compositions described above.

These and other aspects and advantages of the present invention will become apparent from the subsequent detailed description and the appended claims. It is to be understood that one, some, or all of the properties of the various embodiments described herein may be combined to form other embodiments of the present invention.

### BRIEF DESCRIPTION OF THE DRAWINGS

FIG. 1 depicts a schematic of an exemplary embodiment of the method of preparing a peptide having an N-terminal histidine.

FIG. 2 depicts an SCX HPLC chromatograph of a mixture of D-His Exenatide and Exenatide.

FIG. 3 depicts an SCX HPLC chromatograph of Fmoc-D-His(Trt)-OH.

### DETAILED DESCRIPTION OF THE INVENTION

The present invention discloses a method of preparing a peptide having an N-terminal histidine that reduces racemization of the N-terminal histidine in the solid phase peptide synthesis (SPPS) process. Typically, in an Fmoc-based SPPS, the N-terminal Fmoc group is removed in basic conditions prior to removal of the side chain protection groups and cleavage of the peptide from the resin under acidic conditions. The on-resin deblock reaction allows highly efficient purification of the peptide-resin intermediate by simply washing side products and unreacted reagents off the resin, thereby ensuring a high overall yield of the synthesis scheme. However, inventors of the present application discovered that exposure of Fmoc-protected N-terminal His of the peptide to basic conditions in the deblock step contributes to the significant level of N-terminal His racemization, and the high content of impurity having the incorrect N-terminal His enantiomer in the final product. A basic in-solution deblock step following acidic cleavage of Fmoc-protected peptides off the resin is used in the methods of the present application. The inventors found surprisingly that an additional purification step is not required prior to the in-solution deblock step, and the crude peptide produced from the method contains a low content of impurity having the incorrect N-terminal His enantiomer even before any purification. Methods disclosed in the present application provide a cost-efficient and scalable solid phase synthesis scheme for producing peptide drug compositions with enantiomerically pure N-terminal histidine, including, for example, Exenatide and Lixisenatide, which are useful for treating diabetes.

Accordingly, in some embodiments, there is provided a method of preparing a peptide having an N-terminal histidine, comprising: (a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the peptide having the N-terminal histidine.

Further provided is a composition comprising a plurality of resin-free peptide intermediates, wherein each resin-free peptide intermediate comprises an Fmoc-protected N-terminus and unprotected amino acid side chains.

### I. Definition

Unless defined otherwise, all technical and scientific terms used herein have the same meaning as commonly understood by one of ordinary skill in the art to which this invention belongs.

Unless otherwise stated, all chiral amino acids in naturally occurring peptides and synthetic analogs thereof (such as Exenatide, Lixisenatide, and Liraglutide) are in the L-configuration.

As used herein, “deblock” refers to removal of the protection group of the amine group (—NH₂) at the N-terminus of a peptide attached to a resin during solid phase peptide synthesis. In reference to deblock an Fmoc group, the reaction is also referred to as “deFmoc”.

As used herein, “resin-bound peptide intermediate” refers to a peptide having its C-terminal carboxyl group attached to a resin (for example, via a linker), and its N-terminal amino group protected by an Fmoc group. Side chains of amino acids in the resin-bound peptide intermediate that are reactive under the reaction conditions (such as acidic cleavage step or basic deblock step) in the methods described herein may further be protected by suitable protection groups. The resin-bound peptide intermediate has the full amino acid sequence of the peptide having the N-terminal histidine.

As used herein, “resin-free peptide intermediate” refers to a peptide that is not attached to a resin, and its N-terminal amino group is protected by an Fmoc group. The resin-free peptide intermediate may have no protection groups attached to the side chains of the amino acids in the resin-free peptide intermediate. The resin-free peptide intermediate has the full amino acid sequence of the peptide having the N-terminal histidine.

As used herein, “treatment” or “treating” is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., delaying the worsening of the disease), delaying the spread of the disease, reducing recurrence rate of the disease, delay or slowing the progression of the disease, ameliorating the disease state, providing a remission (partial or total) of the disease, decreasing the dose of one or more other medications required to treat the disease, delaying the progression of the disease, increasing the quality of life, and/or prolonging survival. The methods of the invention contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms. An effective amount can be administered in one or more administrations.

As used herein, an “individual” or a “subject” refers to a mammal, including, but not limited to, human, bovine, horse, feline, canine, rodent, or primate. In some embodiments, the individual is a human.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

As used herein and in the appended claims, the singular forms “a,” “or,” and “the” include plural referents unless the context clearly dictates otherwise.

Abbreviations:

ACN acetonitrile

AA Amino acid

Boc Tert-butoxycarbonyl

Cl-HOBt 6-chloro-1-hydroxybenzotriazole

DCM Dichloromethane

DIC Diisopropylcarbodiimide

DMF N,N-dimethyl formamide

EDT Ethanedithiol

eq equivalent

Fmoc 9-fluorenylmethyloxycarbonyl

His Histidine

HOBt 1-hydroxybenzotrizole

HPLC High performance liquid chromatography

hr(s) Hour(s)

isoleucine

min minutes

mmt 4-methoxytrityl

mtt 4-Methyltrityl

Pip Piperidine

SCX-HPLC Strong cation-exchange chromatography

TEA Trifluoroacetic acid

TIS Triisopropylsilane

Trt Trityl

### II. Methods of Preparation

The present invention provides a method of preparing a peptide having an N-terminal histidine (such as L-His), comprising: (a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, the peptide having the N-terminal histidine is Exenatide. In some embodiments, the peptide having the N-terminal histidine is Lixisenatide. In some embodiments, the peptide having the N-terminal histidine is Liraglutide.

In some embodiments, there is provided a method of preparing a peptide having an N-terminal histidine (such as L-His), comprising: (a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; (b) contacting the resin-free peptide intermediate with a basic deblock solution to remove the Fmoc group from the N-terminus of the resin-free peptide intermediate to provide a reaction mixture comprising the peptide having the N-terminal histidine; and (c) purifying the peptide having the N-terminal histidine (such as using chromatography) from the reaction mixture to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, the peptide having the N-terminal histidine is Exenatide. In some embodiments, the peptide having the N-terminal histidine is Lixisenatide. In some embodiments, the peptide having the N-terminal histidine is Liraglutide.

In some embodiments, there is provided a method of preparing a peptide having an N-terminal histidine (such as L-His), comprising: (a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; (b) contacting the resin-free peptide intermediate with a basic deblock solution to remove the Fmoc group from the N-terminus of the resin-free peptide intermediate to provide a reaction mixture comprising the peptide having the N-terminal histidine; (c) contacting the reaction mixture with an acidic neutralization solution; and (d) purifying the peptide having the N-terminal histidine (such as using chromatography) from the reaction mixture to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, the acidic neutralization solution comprises TFA (such as 50% TFA in water). In some embodiments, the peptide having the N-terminal histidine is Exenatide. In some embodiments, the peptide having the N-terminal histidine is Lixisenatide. In some embodiments, the peptide having the N-terminal histidine is Liraglutide.

In some embodiments, there is provided a method of preparing a peptide having an N-terminal histidine (such as L-His), comprising: (a) synthesizing a resin-bound peptide intermediate on a resin using Fmoc-protected amino acids according to the sequence of the peptide having the N-terminal histidine; (b) contacting the resin-bound peptide intermediate with an acidic cleavage solution to provide a resin-free peptide intermediate, wherein the N-terminus of the resin-bound peptide intermediate is protected by an Fmoc group; and (c) contacting the resin-free peptide intermediate with a basic deblock solution to remove the Fmoc group from the N-terminus of the resin-free peptide intermediate to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the peptide having the N-terminal histidine is Exenatide. In some embodiments, the peptide having the N-terminal histidine is Lixisenatide. In some embodiments, the peptide having the N-terminal histidine is Liraglutide.

In some embodiments, there is provided a method of preparing a peptide having an N-terminal histidine (such as L-His), comprising: (a) synthesizing a resin-bound peptide intermediate on a resin using Fmoc-protected amino acids according to the sequence of the peptide having the N-terminal histidine; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising the peptide having the N-terminal histidine; and (d) purifying the peptide having the N-terminal histidine (such as using chromatography) from the reaction mixture to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the peptide having the N-terminal histidine is Exenatide. In some embodiments, the peptide having the N-terminal histidine is Lixisenatide. In some embodiments, the peptide having the N-terminal histidine is Liraglutide.

In some embodiments, there is provided a method of preparing a peptide having an N-terminal histidine (such as L-His), comprising: (a) synthesizing a resin-bound peptide intermediate on a resin using Fmoc-protected amino acids according to the sequence of the peptide having the N-terminal histidine; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising the peptide having the N-terminal histidine; (d) contacting the reaction mixture with an acidic neutralization solution; and (e) purifying the peptide having the N-terminal histidine (such as using chromatography) from the reaction mixture to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, the peptide having the N-terminal histidine is Exenatide. In some embodiments, the peptide having the N-terminal histidine is Lixisenatide. In some embodiments, the peptide having the N-terminal histidine is Liraglutide.

### Acidic Cleavage and Basic Deblock Steps

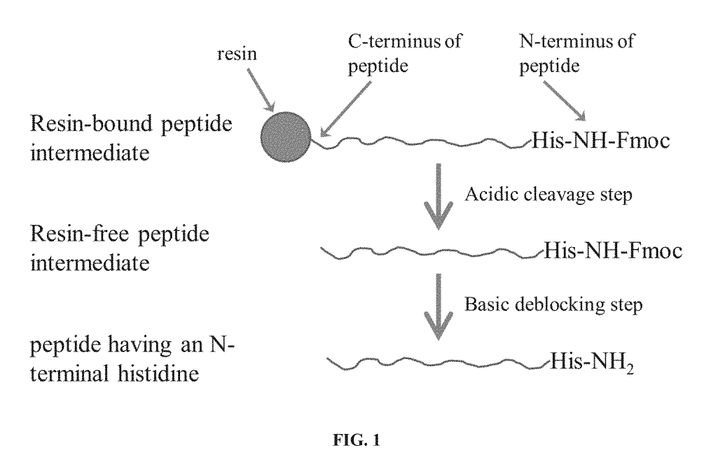

The methods of the present application comprises cleavage of the resin-bound peptide intermediate under acidic conditions to provide a resin-free peptide intermediate, followed by a basic deblock step to remove the Fmoc protection group from the resin-free peptide intermediate. An exemplary method comprising the acidic cleavage and basic deblock steps is illustrated in FIG. 1.

The resin-bound peptide intermediate may be cleaved from the resin using any suitable acid known in the art, including, but not limited to, trifluoroacetic (TFA) acid, trifluoromethanesulfonic acid, hydrogen bromide, hydrogen chloride, hydrogen fluoride, etc. In some embodiments, the acidic cleavage solution further comprises one or more scavengers, including, but not limited to, ethanedithiol (EDT), triisopropylsilane (TIS), phenol, and thioanisole. Reagents and cleavage conditions known in the art are described, for example, in “Introduction to Cleavage Techniques, Applied Biosystems, Inc.,” 1990, pp. 6-12, which is incorporated herein by reference. In some embodiments, the cleavage step cleaves the resin-bound peptide intermediate off the resin, as well as the side chain protection groups of the amino acids in the peptide intermediate. The cleavage reagents and conditions used herein do not affect the Fmoc protection group on the N-terminus of the peptide intermediate. Thus, a resin-free peptide intermediate having its N-terminus protected by an Fmoc group is provided after the acidic cleavage step.

In some embodiments, the acidic cleavage solution comprises TFA. In some embodiments, the acidic cleavage solution comprises water. In some embodiments, the acidic cleavage solution comprises EDT. In some embodiments, the acidic cleavage solution comprises TIS. In some embodiments, the acidic cleavage solution comprises TFA, EDT, TIS and water.

Suitable concentrations of TFA in the acidic cleavage solution include, but are not limited to, at least about any of 90%, 91%, 92%, 93%, 94%, 95%, 96%, 97%, 98%, or more as measured by volume. In some embodiments, the acidic cleavage solution comprises about 94% of TFA by volume.

Suitable concentration of a scavenger (such as EDT or TIS) in the acidic cleavage solution include, but are not limited to, no more than about any of 10%, 9%, 8%, 7%, 6%, 5%, 4%, 3%, 2.5%, 2%, 1.5%, 1% or less. In some embodiments, the acidic cleavage solution comprises about 2% of EDT by volume. In some embodiments, the acidic cleavage solution comprises about 2% of TIS by volume. In some embodiments, the acidic cleavage solution comprises about 2% of EDT and about 2% of TIS by volume.

Suitable concentration of water in the acidic cleavage solution include, but are not limited to, no more than about any of 10%, 9%, 8%, 7%, 6%, 5%, 4%, 3%, 2.5%, 2%, 1.5%, 1% or less. In some embodiments, the acidic cleavage solution comprises about 2% of water by volume. In some embodiments, the acidic cleavage solution comprises about 94% of TFA, 2% of EDT, 2% of TIS and about 2% of water by volume.

The resin-bound peptide intermediate may be contacted with the acid cleavage solution at any suitable temperature (such as room temperature) and for any suitable period of time. In some embodiments, the resin-bound peptide intermediate is contacted with the acidic cleavage solution for at least about any of 1 hour, 2 hours, 3 hours, 4 hours, 5 hours, 6 hours, 7 hours, 8 hours, 12 hours, or more. In some embodiments, the resin-bound peptide intermediate is contacted with the acidic cleavage solution for no more than about any of 12 hours, 8 hours, 7 hours, 6 hours, 5 hours, 4 hours, 3 hours, 2 hours, 1 hour, or less. In some embodiments, the resin-bound peptide intermediate is contacted with the acidic cleavage solution for any of about 1 hour to about 4 hours, about 2 hours to about 3 hours, about 3 hours to about 4 hours, about 4 hours to about 5 hours, about 5 hours to about 6 hours, about 6 hours to about 8 hours, about 8 hours to about 12 hours, about 2 hours to about 4 hours, about 4 hours to about 6 hours, or about 2 hours to about 5 hours.

In some embodiments, the resin-free peptide intermediate is isolated from the reaction mixture after the acidic cleavage step. For example, any solid phase materials, such as resin or resin-bound peptide may be separated from the solution comprising the resin-free peptide intermediate by filtration or centrifugation to provide a crude mixture. TFA and other volatile reagents may be removed from the crude mixture by rotavaporation and/or lyophilization. In some embodiments, the resin-free peptide intermediate may be precipitated from the crude mixture using ether. In some embodiments, the resin-free peptide intermediate is purified from the crude mixture by chromatography (such as HPLC). Exemplary chromatography methods for purifying peptides include, but are not limited to, reverse-phase chromatography (such as using C4, C8 or C18 column), ion exchange chromatography, and size exclusion chromatography. In some embodiments, the resin-free peptide intermediate is purified from the crude mixture on a C18 column. In some embodiments, the resin-free peptide intermediate is not purified from the crude mixture prior to the deblock step. In some embodiments, the crude mixture comprising the resin-free peptide intermediate is contacted with the basic deblock solution. The inventors surprisingly found that purification of the resin-free peptide intermediate from the crude mixture obtained after the acidic cleavage reaction is not a required step of the methods. Inclusion of an additional purification step after the acidic cleavage reaction may decrease the overall yield and increase the cost of the synthesis scheme.

The Fmoc protection group may be removed from the N-terminus of the resin-free intermediate peptide in solution using any suitable base to provide the peptide having the N-terminal histidine. In some embodiments, the resin-free intermediate peptide is contacted with a basic deblock solution comprising piperidine to remove the Fmoc group from the N-terminus of the resin-free peptide intermediate. In some embodiments, the basic deblock solution comprises water. In some embodiments, the basic deblock solution comprises ACN. In some embodiments, the basic deblock solution comprises piperidine in water and ACN.

A suitable concentration of the piperidine in the basic deblock solution includes, but is not limited to, no more than about any of 30%, 29%, 28%, 27%, 26%, 24%, 23%, 22%, 21%, 20%, 19%, 18%, 17%, 16%, 15%, 14%, 13%, 12%, 11%, or 10%. In some embodiments, the concentration of the piperidine in the basic deblock solution is any of about 10% to about 15%, about 15% to about 16%, about 16% to about 17%, about 17% to about 18%, about 18% to about 19%, about 19% to about 20%, about 20% to about 21%, about 21% to about 22%, about 22% to about 23%, about 23% to about 24%, about 24% to about 25%, about 15% to about 20%, about 20% to about 25%, about 25% to about 30%, about 15% to about 25%, about 18% to about 22%, or about 10% to about 30%.

The resin-free peptide intermediate may be contacted with the basic deblock solution under any suitable temperature, such as at room temperature, at about 20° C., about 15° C., about 10° C., or about 4° C. The resin-free peptide intermediate may be contacted with the basic deblock solution for any suitable period of time. In some embodiments, the resin-free peptide intermediate is contacted with the basic deblock solution for no more than about any of 1 hour, 50 minutes, 45 minutes, 40 minutes, 35 minutes, 30 minutes, 25 minutes, 20 minutes, 15 minutes, 10 minutes, or less. In some embodiments, the resin-free peptide intermediate is contacted with the basic deblock solution for any of about 45 minutes to about 1 hour, about 30 minutes to about 45 minutes, about 15 minutes to about 20 minutes, about 20 minutes to about 25 minutes, about 25 minutes to about 30 minutes, about 15 minutes to about 25 minutes, about 20 minutes to about 30 minutes, or about 15 minutes to about 30 minutes.

In some embodiments, the reaction mixture comprising the peptide having the N-terminal histidine is contacted with an acidic neutralization solution after the deblock step. The acidic neutralization solution can neutralize the basic deblock agent (such as piperidine) in the reaction mixture. The neutralization step can reduce the exposure of the peptide having the N-terminal histidine to basic conditions, which may contribute to racemization of the N-terminal histidine. In some embodiments, the reaction mixture is contacted with one or more equivalents (with respect to the basic deblock agent, such as piperidine) of an acidic neutralization solution. In some embodiments, the final pH of the reaction mixture after contacting with the acidic neutralization solution is no more than about any of 4, 5, 6, 7, or 7.5. Any suitable acid may be used in the acidic neutralization solution. In some embodiments, the acidic neutralization solution comprises TFA. In some embodiments, the acidic neutralization solution is an aqueous solution of TFA. Suitable concentrations of the TFA in the neutralization solution include, but are not limited to, at least about any of 10%, 20%, 30%, 40%, 50%, 60%, 70%, 80%, 90%, 95%, or more by volume. In some embodiments, the acidic neutralization solution comprises 50% of TFA by volume, for example, in an aqueous solution.

### Solid-Phase Synthesis

The methods of the present application may comprise one or more solid phase peptide synthesis (SPPS) steps to prepare the resin-bound peptide intermediate having an N-terminal Fmoc protection group. Standard Fmoc-based solid-phase peptide synthesis techniques may be used for the synthesis. In some embodiments, the method comprises synthesizing the resin-bound peptide intermediate on a resin using Fmoc-protected amino acids according to the sequence of the peptide having the N-terminal histidine. In some embodiments, an automated or semiautomated peptide synthesizer is used to perform the SPPS.

Standard Fmoc-based SPPS methods have been described in the art; see, for example, Chan, W C & White, P D, Fmoc solid phase peptide synthesis: A practical approach. Oxford University Press, 2004, which is incorporated herein by reference. The coupling and deblock steps for the attachment of each amino acid to the growing peptide chain are performed by methods known to the person skilled in the art taking into consideration the Fmoc protection strategy and the selected solid phase material. Peptide bonds may be formed using the various activation procedures known to the person skilled in the art, eg., by reacting a C-terminal activated derivative (acid halide, acid anhydride, activated ester e.g., HOBt-ester, etc.) of the appropriate amino acid or peptide with the amino group of the relevant amino acid or peptide as known to a person skilled in peptide chemistry. The SPPS may be carried out in any suitable solvent, such as DMF.

In some embodiments, using automated or semi-automated peptide synthesis techniques, an α-N-Fmoc protected amino acid and an amino acid attached to the growing peptide chain on a resin are coupled at room temperature in an inert solvent such as dimethylformamide, N-methylpyrrolidinone or methylene chloride in the presence of coupling agents such as dicyclohexylcarbodiimide and 1-hydroxybenzotriazole in the presence of a base such as diisopropylethylamine. After each coupling step, the α-N-Fmoc protecting group is removed from the resulting peptide-resin using a reagent such as piperidine in a deblock step, and the coupling reaction is repeated with the next desired N-Fmoc protected amino acid to be added to the peptide chain.

Importantly, in the methods of the present application, the resin-bound peptide intermediate comprising the N-terminal Fmoc-protected histidine is not subjected to the normal on-resin deblock step that removes the α-N-Fmoc protecting group on the N-terminal histidine. In some embodiments, the coupling agents used in the coupling step for the N-terminal histidine comprise DIC (such as about 3 to about 8 eq.) and HOBt (such as about 3 to about 8 eq.). In some embodiments, the coupling steps are carried out at about 15° C. to about 30° C.

The solvents, coupling or deblock reagents, Fmoc-protected amino acid derivatives, the resin, and automated peptide synthesizer used in the solid phase peptide synthesis steps are known in the art and available from commercial sources. Examples of suitable solid phase support materials include, but are not limited to, functionalized resins such as polystyrene, polyacrylamide, polydimethylacrylamide, polyethyleneglycol, cellulose, polyethylene, polyethyleneglycol grafted on polystyrene, latex, DYNABEADS™, etc. The resin may further be connected to the C-terminus of the peptide via a linker, such as 2,4-dimethoxy-4′-hydroxy-benzophenone, 4-(4-hydroxy-methyl-3-methoxyphenoxy)-butyric acid, 4-hydroxy-methylbenzoic acid, 4-hydroxymethyl-phenoxyacetic acid, 3-(4-hydroxymethylphenoxy)propionic acid, and p-[(R,S)-a[1-(9H-fluoren-9-yl)methoxy formamido]-2,4-dimethoxybenzyl]-phenoxy-acetic acid. In some embodiments, an Fmoc Rink amide (RAM) resin, such as a resin having a 4′-{(R,S)-alpha-[1-(9-Fluorenyl)methoxycarbonylamino]-2,4-dimethoxybenzyl}-phenoxyacetic acid linker, is used in the SPPS. In some embodiments, an HMP resin, such as a resin having a 4-(Hydroxymethyl)phenoxyacetic acid linker is used in the SPPS.

Furthermore, it may be necessary or desirable to include side-chain protection groups when using amino acid residues carrying functional groups which are reactive under the prevailing conditions. The necessary protection scheme will be known to the person skilled in the art (see, for example, M Bodanszky and A Bodanszky, “The Practice of Peptide Synthesis”, 2 Ed, Springer-Verlag, 1994, J Jones, “The Chemical Synthesis of Peptides”, Clarendon Press, 1991, and Dryland et al, 1986, J Chem Soc. Perkin Trans 1 125-137). Exemplary N-Fmoc and side-chain protected amino acids building blocks may include, but not limited to, Fmoc-Arg(Pmc), Arg(Pbf), Fmoc-Thr(tBu), Fmoc-Ser(tBu), Fmoc-Tyr(tBu), Fmoc-Lys(Boc), Fmoc-Glu(OtBu), Fmoc-His(Trt), Fmoc-Asn(Trt), and Fmoc-Gln(Trt), Cys(Trt), Trp(Boc), Asp(OtBu). In some embodiments, the side chain of the N-terminal histidine of the resin-bound peptide intermediate is protected by a group selected from trityl (Trt), 4-methyltrityl (Mtt), and p-methoxytrityl (Mmt).

### Purification

The methods of the present application may comprise one or more purification steps. For example, the resin-free peptide intermediate, and/or the reaction mixture after the deblock step comprising the peptide having the N-terminal histidine may be purified. Peptides may be purified by column chromatography (such as HPLC, analytical or preparative scale), including, but not limited to, reverse-phase chromatography (such as using C4, C8 or C18 column), ion exchange chromatography, and size exclusion chromatography. In some embodiments, the method comprises purifying the peptide having the N-terminal histidine using a C18 column (such as preparative column) after the deblock step. The mobile phase can be set up using two buffers, for example, MPA buffer is 0.1% TFA in water, and MPB buffer is 100% ACN. The buffers may be delivered to the analytical column at a flow rate of about 1.0 ml/min, and to the preparative column at about 15 ml/min. The samples comprising peptides to be purified may be filtered, such as through a 1μ filter, prior to loading onto the column.

The peptide having the N-terminal histidine can be analyzed using methods known in the art, including, but not limited to, amino acid analysis, mass spectrometry (such as GC/MS, or MALDI-TOF), and analytical column chromatography (such as SCX-HPLC, or HPLC using an analytical C18 column). For example, amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen, et al., The Pico Tag Method: A Manual of Advanced Techniques for Amino Acid Analysis, pp. 11-52, Millipore Corporation, Milford, Mass. (1989)). Fast atom bombardment analysis may be carried out by M-Scan, Incorporated (West Chester, Pa.). Mass calibration may be performed using cesium iodide or cesium iodide/glycerol. Plasma desorption ionization analysis using time of flight detection may be carried out on an Applied Biosystems Bio-Ion 20 mass spectrometer. Electrospray mass spectroscopy may be carried and on a VG-Trio machine. In some embodiments, the level of racemization of the N-terminal histidine in the peptide is determined using SCX-HPLC, for example, using the methods described in Example 1.

### Exemplary Peptides

The methods described herein are applicable to the synthesis of any peptides having an N-terminal histidine. Exemplary peptides of particular interest include, but are not limited to, Exenatide (e.g., peptides comprising the amino acid sequence of SEQ ID NO:1), Lixisenatide (e.g., peptides comprising the amino acid sequence of SEQ ID NO:2), and Liraglutide (e.g., peptides comprising the amino acid sequence of SEQ ID NO:3).

Thus, in some embodiments, there is provided a method of preparing Exenatide, comprising: (a) contacting a resin-bound peptide intermediate having the amino acid sequence of Exenatide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide Exenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Exenatide comprises the amino acid sequence of SEQ ID NO:1. In some embodiments, Exenatide has the amino acid sequence of SEQ ID NO:1.

In some embodiments, there is provided a method of preparing Exenatide, comprising: (a) optionally synthesizing a resin-bound peptide intermediate on a resin using Fmoc-protected amino acids according to the sequence of Exenatide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Exenatide; (d) optionally contacting the reaction mixture with an acidic neutralization solution; and (e) optionally purifying Exenatide (such as using chromatography) from the reaction mixture to provide Exenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Exenatide comprises the amino acid sequence of SEQ ID NO:1. In some embodiments, Exenatide has the amino acid sequence of SEQ ID NO:1.

In some embodiments, there is provided a method of preparing Lixisenatide, comprising: (a) contacting a resin-bound peptide intermediate having the amino acid sequence of Lixisenatide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide Lixisenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Lixisenatide comprises the amino acid sequence of SEQ ID NO:2. In some embodiments, Lixisenatide has the amino acid sequence of SEQ ID NO:2.

In some embodiments, there is provided a method of preparing Lixisenatide, comprising: (a) optionally synthesizing a resin-bound peptide intermediate on a resin using Fmoc-protected amino acids according to the sequence of Lixisenatide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Lixisenatide; (d) optionally contacting the reaction mixture with an acidic neutralization solution; and (e) optionally purifying Lixisenatide (such as using chromatography) from the reaction mixture to provide Lixisenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Lixisenatide comprises the amino acid sequence of SEQ ID NO:2. In some embodiments, Lixisenatide has the amino acid sequence of SEQ ID NO:2.

In some embodiments, there is provided a method of preparing Liraglutide, comprising: (a) contacting a resin-bound peptide intermediate having the amino acid sequence of Liraglutide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide Liraglutide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Liraglutide comprises the amino acid sequence of SEQ ID NO:3. In some embodiments, Liraglutide has the amino acid sequence of SEQ ID NO:3.

In some embodiments, there is provided a method of preparing Liraglutide, comprising: (a) optionally synthesizing a resin-bound peptide intermediate on a resin using Fmoc-protected amino acids according to the sequence of Liraglutide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Liraglutide; (d) optionally contacting the reaction mixture with an acidic neutralization solution; and (e) optionally purifying Liraglutide (such as using chromatography) from the reaction mixture to provide Liraglutide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Liraglutide comprises the amino acid sequence of SEQ ID NO:3. In some embodiments, Liraglutide has the amino acid sequence of SEQ ID NO:3.

(Exenatide)
SEQ ID NO: 1
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Pro-Ser-NH₂ (amide group attached to the C-
terminus)
(Lixisenatide)
SEQ ID NO: 2
His-Gly-Glu-Gly-Thr-Phe-Thr-Ser-Asp-Leu-Ser-Lys-
Gln-Met-Glu-Glu-Glu-Ala-Val-Arg-Leu-Phe-Ile-Glu-
Trp-Leu-Lys-Asn-Gly-Gly-Pro-Ser-Ser-Gly-Ala-Pro-
Pro-Ser-Lys-Lys-Lys-Lys-Lys-Lys-NH₂ (amide group
attached to the C-terminus)
(Liraglutide)
SEQ ID NO: 3
His-Ala-Glu-Gly-Thr-Phe-Thr-Ser-Asp-Val-Ser-Ser-
Tyr-Leu-Glu-Gly-Gln-Ala-Ala-Lys(y-Glu-Palm)-Glu-
Phe-Ile-Ala-Trp-Leu-Val-Arg-Gly-Arg-Gly
(unmodified carboxylic acid group at the C-
terminus)

### Large-Scale Manufacture Process

The methods described herein can be used to produce peptides having an N-terminal histidine, such as Exenatide, Lixisenatide, or Liraglutide, or compositions thereof at a large scale, such as industrial scale.

Thus, in some embodiments, there is provided a large-scale process for manufacturing a peptide having an N-terminal histidine (such as L-His), comprising: (a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, the process is automated. In some embodiments, the peptide having the N-terminal histidine is Exenatide (such as a peptide comprising the amino acid sequence of SEQ ID NO:1). In some embodiments, the peptide having the N-terminal histidine is Lixisenatide (such as a peptide comprising the amino acid sequence of SEQ ID NO:2). In some embodiments, the peptide having the N-terminal histidine is Liraglutide (such as a peptide comprising the amino acid sequence of SEQ ID NO:3).

In some embodiments, there is provided a large-scale process for manufacturing a peptide having an N-terminal histidine (such as L-His), comprising: (a) optionally synthesizing a resin-bound peptide intermediate on a resin using Fmoc-protected amino acids according to the sequence of the peptide having the N-terminal histidine; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising the peptide having the N-terminal histidine; (d) optionally contacting the reaction mixture with an acidic neutralization solution; and (e) optionally purifying the peptide having the N-terminal histidine (such as using chromatography) from the reaction mixture to provide the peptide having the N-terminal histidine. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, the process is automated. In some embodiments, the peptide having the N-terminal histidine is Exenatide (such as a peptide comprising the amino acid sequence of SEQ ID NO:1). In some embodiments, the peptide having the N-terminal histidine is Lixisenatide (such as a peptide comprising the amino acid sequence of SEQ ID NO:2). In some embodiments, the peptide having the N-terminal histidine is Liraglutide (such as a peptide comprising the amino acid sequence of SEQ ID NO:3).

The scale of the process described herein is at least about any of 1, 2, 5, 10, 30, 40, 50, 60, 70, 80, 90, 100, 150, 200, 250, 300, 350, 400, 450, 500, 550, 600, 650, 700, 750, 800, 850, 900, 1000, 1500, 2000, 2500, 3000, 3500, 4000, 4500, 5000, or 10000 grams by the amount of the peptides having the N-terminal histidine produced in the process. In some embodiments, the amount of the starting materials (such as amino acid derivatives, resin, resin-bound peptide intermediate, or resin-free peptide intermediate) is at least about any of 0.01, 0.02, 0.05, 0.1, 0.2, 0.5, 1, 2, 5, 10, 30, 40, 50, 60, 70, 80, 90, 100, 150, 200, 250, 300, 350, 400, 450, 500, 550, 600, 650, 700, 750, 800, 850, 900, 1000, 1500, 2000, 2500, 3000, 3500, 4000, 4500, 5000, or 10000 grams. In some embodiments, the volume of the reaction mixtures (such as in the SPPS steps, acidic cleavage step, or deblock step) is at least about any of 100 mL, 200 mL, 300 mL, 400 mL, 500 mL, 600 mL, 700 mL, 800 mL, 900 mL, 1 L, 1.5 L, 2 L, 2.5 L, 3 L, 4 L, 5 L, 6 L, 7 L, 8 L, 9 L, 10 L, 20 L, 30 L, 40 L, 50 L, 60 L, 70 L, 80 L, 90 L, 100 L or more. In some embodiments, the volume of the acidic cleavage reaction mixture in a large-scale manufacture process is at least about 100 L. In some embodiments, the volume of the basic deblock reaction mixture in a large-scale manufacture process is at least about 10 L.

Further provided are systems for carrying out any one of the methods described herein. In some embodiments, the system comprises an automated or semiautomated peptide synthesizer. In some embodiments, the system further comprises an HPLC, such as SCX-HPLC. In some embodiments, the system is for large-scale manufacture.

### III. Compositions and Methods of Treatment

Further provided by the present application are peptides prepared using any of the methods described herein, and compositions (such as pharmaceutical compositions) comprising a plurality of peptides prepared by any of the methods described herein. In some embodiments, the plurality of peptides has substantially the same amino acid sequence.

Thus, in some embodiments, there is provided a composition (such as a pharmaceutical composition) comprising a plurality of peptides having an N-terminal histidine, wherein the percentage of peptides having an N-terminal D-histidine in the composition is less than about 1% (such as less than about any of 0.75%, 0.5%, or 0.3%), and wherein the plurality of peptides is prepared by steps comprising: (a) contacting a resin-bound peptide intermediate having the amino acid sequence of the peptides with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the plurality of peptides. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b).

In some embodiments, there is provided a composition (such as a pharmaceutical composition) comprising a plurality of peptides having an N-terminal histidine, wherein the percentage of peptides having an N-terminal D-histidine in the composition is less than about 1% (such as less than about any of 0.75%, 0.5%, or 0.3%), and wherein the plurality of peptides is prepared by steps comprising: (a) optionally synthesizing a resin-bound peptide intermediate on a resin using Fmoc-protected amino acids according to the sequence of the peptides; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising the peptides; (d) optionally contacting the reaction mixture with an acidic neutralization solution; and (e) optionally purifying the plurality of peptides (such as using chromatography) from the reaction mixture to provide the plurality of peptides. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water).

In some embodiments, there is provided a composition (such as a pharmaceutical composition) comprising a plurality of Exenatide, wherein the percentage of Exenatide having an N-terminal D-histidine in the composition is less than about 1% (such as less than about any of 0.75%, 0.5%, or 0.3%), and wherein the plurality of Exenatide is prepared by steps comprising: (a) contacting a resin-bound peptide intermediate having the amino acid sequence of Exenatide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the plurality of Exenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Exenatide comprises the amino acid sequence of SEQ ID NO:1. In some embodiments, Exenatide has the amino acid sequence of SEQ ID NO:1.

In some embodiments, there is provided a composition (such as a pharmaceutical composition) comprising a plurality of Exenatide, wherein the percentage of Exenatide having an N-terminal D-histidine in the composition is less than about 1% (such as less than about any of 0.75%, 0.5%, or 0.3%), and wherein the plurality of Exenatide is prepared by steps comprising: (a) optionally synthesizing a resin-bound peptide intermediate on a resin using Fmoc-protected amino acids according to the sequence of Exenatide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Exenatide; (d) optionally contacting the reaction mixture with an acidic neutralization solution; and (e) optionally purifying Exenatide (such as using chromatography) from the reaction mixture to provide the plurality of Exenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Exenatide comprises the amino acid sequence of SEQ ID NO:1. In some embodiments, Exenatide has the amino acid sequence of SEQ ID NO:1.

In some embodiments, there is provided a composition (such as a pharmaceutical composition) comprising a plurality of Lixisenatide, wherein the percentage of Lixisenatide having an N-terminal D-histidine in the composition is less than about 1% (such as less than about any of 0.75%, 0.5%, or 0.3%), and wherein the plurality of Lixisenatide is prepared by steps comprising: (a) contacting a resin-bound peptide intermediate having the amino acid sequence of Lixisenatide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the plurality of Lixisenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Lixisenatide comprises the amino acid sequence of SEQ ID NO:2. In some embodiments, Lixisenatide has the amino acid sequence of SEQ ID NO:2.

In some embodiments, there is provided a composition (such as a pharmaceutical composition) comprising a plurality of Lixisenatide, wherein the percentage of Lixisenatide having an N-terminal D-histidine in the composition is less than about 1% (such as less than about any of 0.75%, 0.5%, or 0.3%), and wherein the plurality of Lixisenatide is prepared by steps comprising: (a) optionally synthesizing a resin-bound peptide intermediate on a resin using Fmoc-protected amino acids according to the sequence of Lixisenatide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Lixisenatide; (d) optionally contacting the reaction mixture with an acidic neutralization solution; and (e) optionally purifying Lixisenatide (such as using chromatography) from the reaction mixture to provide the plurality of Lixisenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Lixisenatide comprises the amino acid sequence of SEQ ID NO:2. In some embodiments, Lixisenatide has the amino acid sequence of SEQ ID NO:2.

In some embodiments, there is provided a composition (such as a pharmaceutical composition) comprising a plurality of Liraglutide, wherein the percentage of Liraglutide having an N-terminal D-histidine in the composition is less than about 1% (such as less than about any of 0.75%, 0.5%, or 0.3%), and wherein the plurality of Liraglutide is prepared by steps comprising: (a) contacting a resin-bound peptide intermediate having the amino acid sequence of Liraglutide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the plurality of Liraglutide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Liraglutide comprises the amino acid sequence of SEQ ID NO:3. In some embodiments, Liraglutide has the amino acid sequence of SEQ ID NO:3.

In some embodiments, there is provided a composition (such as a pharmaceutical composition) comprising a plurality of Liraglutide, wherein the percentage of Liraglutide having an N-terminal D-histidine in the composition is less than about 1% (such as less than about any of 0.75%, 0.5%, or 0.3%), and wherein the plurality of Liraglutide is prepared by steps comprising: (a) optionally synthesizing a resin-bound peptide intermediate on a resin using Fmoc-protected amino acids according to the sequence of Liraglutide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Liraglutide; (d) optionally contacting the reaction mixture with an acidic neutralization solution; and (e) optionally purifying Liraglutide (such as using chromatography) from the reaction mixture to provide the plurality of Liraglutide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Liraglutide comprises the amino acid sequence of SEQ ID NO:3. In some embodiments, Liraglutide has the amino acid sequence of SEQ ID NO:3.

In some embodiments, there is provided a composition (such as a pharmaceutical composition) comprising a plurality of peptides having an N-terminal histidine, wherein the percentage of peptides having an N-terminal D-histidine in the composition is less than about 1% (such as less than about any of 0.75%, 0.5%, or 0.3%).

In some embodiments, there is provided a composition (such as a pharmaceutical composition) comprising a plurality of Exenatide, wherein the percentage of Exenatide having an N-terminal D-histidine in the composition is less than about 1% (such as less than about any of 0.75%, 0.5%, or 0.3%). In some embodiments, Exenatide comprises the amino acid sequence of SEQ ID NO:1. In some embodiments, Exenatide has the amino acid sequence of SEQ ID NO:1.

In some embodiments, there is provided a composition (such as a pharmaceutical composition) comprising a plurality of Lixisenatide, wherein the percentage of Exenatide having an N-terminal D-histidine in the composition is less than about 1% (such as less than about any of 0.75%, 0.5%, or 0.3%). In some embodiments, Lixisenatide comprises the amino acid sequence of SEQ ID NO:2. In some embodiments, Lixisenatide has the amino acid sequence of SEQ ID NO:2.

In some embodiments, there is provided a composition (such as a pharmaceutical composition) comprising a plurality of Liraglutide, wherein the percentage of Exenatide having an N-terminal D-histidine in the composition is less than about 1% (such as less than about any of 0.75%, 0.5%, or 0.3%). In some embodiments, Liraglutide comprises the amino acid sequence of SEQ ID NO:3. In some embodiments, Liraglutide has the amino acid sequence of SEQ ID NO:3.

The compositions described herein have a low level of impurity having the incorrect N-terminal histidine enantiomer (such as D-His). In some embodiments, the percentage of peptides having the incorrect N-terminal histidine enantiomer (such as D-His) in the composition is less than about any of 2%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, or less prior to any purification step. In some embodiments, the percentage of peptides having an the incorrect N-terminal histidine enantiomer (such as D-His) in the composition is less than about any of 1.0%, 0.9%, 0.8%, 0.7%, 0.6%, 0.5%, 0.4%, 0.3%, 0.2%, or less after purification of the peptides having the N-terminal histidine using chromatography, for example on a reverse-phase column (such as preparative scale, C18 column). In some embodiments, the percentage of peptides having the incorrect N-terminal histidine enantiomer (such as D-His) in the composition is any one of about 1% to about 1.5%, about 1.5% to about 2%, about 0.9% to about 1%, about 0.8% to about 0.9%, about 0.7% to about 0.8%, about 0.6% to about 0.7%, about 0.5% to about 0.6%, about 0.4% to about 0.5%, about 0.3% to about 0.4%, about 0.2% to about 0.3%, about 0.2% to about 0.4%, about 0.4% to about 0.6%, about 0.6% to about 0.8%, about 0.8% to about 1%, about 0.5% to about 1%, about 0.25% to about 0.75%, about 0.2% to about 0.5%, or about 0.2% to about 1%. The percentage of peptides having the incorrect N-terminal histidine enantiomer (such as D-His) in the composition can be determined using any known methods in the art, including, for example, amino acid analysis, GC/MS, or SCX-HPLC.

The compositions comprising a plurality of peptides having an N-terminal histidine (such as Exenatide, Lixisenatide, or Liraglutide) described herein can be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

The description is intended to embrace all salts of the compounds described herein, as well as methods of using such salts of the compounds. In one embodiment, the salts of the compounds comprise pharmaceutically acceptable salts. Pharmaceutically acceptable salts are those salts which can be administered as drugs or pharmaceuticals to humans and/or animals and which, upon administration, retain at least some of the biological activity of the free compound (neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, can also be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, can also be prepared. For lists of pharmaceutically acceptable salts, see, for example, P. H. Stahl and C. G. Wermuth (eds.) “Handbook of Pharmaceutical Salts, Properties, Selection and Use” Wiley-VCH, 2011 (ISBN: 978-3-90639-051-2). Several pharmaceutically acceptable salts are also disclosed in Berge, J. Pharm. Sci. 66:1 (1977).

Exemplary pharmaceutically acceptable salts contemplated herein include acid addition salts, such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids, such as hydrochloric acid, sulfuric acid, phosphoric acid, sulfamic acid, acetic acid, citric acid, lactic acid, tartaric acid, malonic acid, methanesulfonic acid, ethane sulfonic acid, benzene sulfonic acid, p-toluenesulfonic acid, cyclohexyl sulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

In some embodiments, the composition (such as pharmaceutical composition) further comprises a carrier, diluent, or excipient, which may facilitate administration of the composition to an individual in need thereof. Examples of carriers, diluents, and excipients include, but are not limited to, calcium carbonate, calcium phosphate, various sugars such as lactose, or types of starch, cellulose derivatives, gelatin, vegetable oils, polyethylene glycols and physiologically compatible solvents.

If desired, solutions of the above compositions may be thickened with a thickening agent such as methylcellulose. They may be prepared in emulsified form, either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g., a Triton).

The pharmaceutical compositions described herein can be prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

Other pharmaceutically acceptable carriers and their formulation are described in standard formulation treatises, e.g., Remington's Pharmaceutical Sciences by E. W. Martin. See also Wang, Y. J. and Hanson, M. A. “Parenteral Formulations of Proteins and Peptides: Stability and Stabilizers,” Journal of Parenteral Science and Technology, Technical Report No. 10, Supp. 42:2S (1988).

In some embodiments, the pharmaceutical composition is in a dry powder formulation. In some embodiments, the pharmaceutical composition is in a liquid formulation, such as a gel formulation, or a parental liquid formulation. In some embodiments, the pharmaceutical composition is in a solid formulation, such as a tablet.

In some embodiments, the pharmaceutical composition comprises a carbohydrate or polyhydric alcohol iso-osmolality modifier. Suitable polyhydric alcohols include, but are not limited to, sorbitol, mannitol, glycerol, and polyethylene glycols (PEGs, such as PEGs of molecular weight 200, 400, 1450, 3350, 4000, 6000, and 8000). Suitable carbohydrates include, but are not limited to, mannose, ribose, trehalose, maltose, glycerol, inositol, glucose, lactose, galactose, and arabinose. The polyhydric alcohols and carbohydrates can also stabilize the peptides against denaturation caused by elevated temperature and by freeze-thaw or freeze-drying processes. In some embodiments, the carbohydrate does not have an adverse effect on diabetic patients.

In certain embodiments of the invention, especially those embodiments where a formulation is used for injection or other parenteral administration, including the routes listed herein, but also including any other route of administration described herein (such as oral, enteric, gastric, etc.), the formulations and preparations used in the methods of the invention are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

In some embodiments, the pharmaceutical composition comprises an anti-microbial preservative selected from the group consisting of m-cresol, benzyl alcohol, methyl ethyl, propyl and butyl parabens, and phenol.

In some embodiments, the pharmaceutical composition comprises a surfactant, such as polysorbate 80 or other non-ionic detergent.

In some embodiments, the pharmaceutical composition is in a lyophilized form. The active ingredient (i.e., peptides) in the pharmaceutical composition is reasonably stable, with or without adequate buffering capacity to maintain the pH of the solution over the intended shelf life of the reconstituted product. In some embodiments, the pharmaceutical composition comprises a bulking agent, such as carbohydrate or polyhydric alcohol (e.g., as described above), saline, or combination thereof, to facilitate cake formation. The bulking agent may also act as a tonicifer and/or iso-osmolality modifier upon reconstitution to either facilitate stability of the active ingredient and/or lessen the pain on injection. In some embodiments, the pharmaceutical composition comprises a surfactant, which may also benefit the properties of the cake and/or facilitate reconstitution. In some embodiments, the pharmaceutical composition comprises a buffer either in the lyophilized form or in the reconstitution solvent. Suitable buffers include, but are not limited to, an acetate, phosphate, citrate or glutamate buffer either alone or in combination to obtain a pH of the final composition of approximately 3.0 to 7.0, more specifically from about pH 4.0 to about 6.0, or from about 4.0 to 5.0.

In some embodiments, the pharmaceutical composition is in a slow release formulation, such as a repository or “depot” so that therapeutically effective amounts of the preparation are delivered into the bloodstream over many hours or days following transdermal injection or other form of delivery.

In some embodiments, the pharmaceutical composition is in a dissolvable gel or patch form, which may be used to facilitate buccal delivery. The gels may be prepared from various types of starch and/or cellulose derivatives. The compositions (such as pharmaceutical compositions) comprising the plurality of peptides (such as Exenatide, Lixisenatide, or Liraglutide) described herein may be useful for treating a disease or a condition in an individual in need thereof. In some embodiments, the composition is used to treat a disease or condition that benefits from increased insulin sensitivity in the individual. In some embodiments, the composition is used to treat diabetes. In some embodiments, the composition is used to treat type I diabetes. In some embodiments, the composition is used to treat type II diabetes. In some embodiments, the composition is used to treat obesity. In some embodiments, the composition used to cause increased insulin sensitivity in an individual. In some embodiments, the composition used to increase insulin secretion. In some embodiments, the composition used to inhibit glucagon secretion. In some embodiments, the composition used to reduce food intake. In some embodiments, the composition used to inhibit gastric emptying. In some embodiments, the composition used to modulate nutrient absorption.

Thus, in some embodiments, there is provided a method of treating a disease or condition in an individual in need thereof, comprising administering to the individual an effective amount of a composition comprising a plurality of peptides having an N-terminal histidine, wherein the percentage of peptides having an N-terminal D-histidine in the composition is less than about 1% (such as less than about any of 0.75%, 0.5%, or 0.3%), and wherein the plurality of peptides is prepared by steps comprising: (a) contacting a resin-bound peptide intermediate having the amino acid sequence of the peptides with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the plurality of peptides. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b).

In some embodiments, there is provided a method of treating a disease or condition in an individual in need thereof, comprising administering to the individual an effective amount of a composition comprising a plurality of peptides having an N-terminal histidine, wherein the percentage of peptides having an N-terminal D-histidine in the composition is less than about 1% (such as less than about any of 0.75%, 0.5%, or 0.3%), and wherein the plurality of peptides is prepared by steps comprising: (a) optionally synthesizing a resin-bound peptide intermediate on a resin using Fmoc-protected amino acids according to the sequence of the peptides; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising the peptides; (d) optionally contacting the reaction mixture with an acidic neutralization solution; and (e) optionally purifying the plurality of peptides (such as using chromatography) from the reaction mixture to provide the plurality of peptides. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water).

In some embodiments, there is provided a method of treating diabetes (such as type II diabetes) in an individual in need thereof, comprising administering to the individual an effective amount of a composition comprising a plurality of Exenatide, wherein the percentage of Exenatide having an N-terminal D-histidine in the composition is less than about 1% (such as less than about any of 0.75%, 0.5%, or 0.3%), and wherein the plurality of Exenatide is prepared by steps comprising: (a) contacting a resin-bound peptide intermediate having the amino acid sequence of Exenatide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the plurality of Exenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Exenatide comprises the amino acid sequence of SEQ ID NO:1. In some embodiments, Exenatide has the amino acid sequence of SEQ ID NO:1. In some embodiments, the composition is administered subcutaneously. In some embodiments, the composition is administered by subcutaneous injection, such as by using an injection pen. In some embodiments, the composition is administered twice daily. In some embodiments, the composition is administered within about 60 minutes prior to morning and evening meals, or before the two main meals of the day, approximately about 6 hours or more apart. In some embodiments, the composition is administered at a dose of about 5 μg twice daily. In some embodiments, the composition is administered at a dose of about 10 μg twice daily. In some embodiments, the composition is administered as an extended-release injectable suspension. In some embodiments, the composition is administered once weekly. In some embodiments, the composition is administered at a dose of about 2 mg once weekly.

In some embodiments, there is provided a method of treating diabetes (such as type II diabetes) in an individual in need thereof, comprising administering to the individual an effective amount of a composition comprising a plurality of Exenatide, wherein the percentage of Exenatide having an N-terminal D-histidine in the composition is less than about 1% (such as less than about any of 0.75%, 0.5%, or 0.3%), and wherein the plurality of Exenatide is prepared by steps comprising: (a) optionally synthesizing a resin-bound peptide intermediate on a resin using Fmoc-protected amino acids according to the sequence of Exenatide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Exenatide; (d) optionally contacting the reaction mixture with an acidic neutralization solution; and (e) optionally purifying Exenatide (such as using chromatography) from the reaction mixture to provide the plurality of Exenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Exenatide comprises the amino acid sequence of SEQ ID NO:1. In some embodiments, Exenatide has the amino acid sequence of SEQ ID NO:1. In some embodiments, the composition is administered subcutaneously. In some embodiments, the composition is administered by subcutaneous injection, such as by using an injection pen. In some embodiments, the composition is administered twice daily. In some embodiments, the composition is administered within about 60 minutes prior to morning and evening meals, or before the two main meals of the day, approximately about 6 hours or more apart. In some embodiments, the composition is administered at a dose of about 5 μg twice daily. In some embodiments, the composition is administered at a dose of about 10 μg twice daily. In some embodiments, the composition is administered as an extended-release injectable suspension. In some embodiments, the composition is administered once weekly. In some embodiments, the composition is administered at a dose of about 2 mg.

In some embodiments, there is provided a method of treating diabetes (such as type II diabetes) in an individual in need thereof, comprising administering to the individual an effective amount of a composition comprising a plurality of Lixisenatide, wherein the percentage of Lixisenatide having an N-terminal D-histidine in the composition is less than about 1% (such as less than about any of 0.75%, 0.5%, or 0.3%), and wherein the plurality of Lixisenatide is prepared by steps comprising: (a) contacting a resin-bound peptide intermediate having the amino acid sequence of Lixisenatide with an acidic cleavage solution to provide a resin-free peptide intermediate, wherein the N-terminus of the resin-bound peptide intermediate is protected by an Fmoc group; and (b) contacting the resin-free peptide intermediate with a basic deblock solution to remove the Fmoc group from the N-terminus of the resin-free peptide intermediate to provide the plurality of Lixisenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (a) provides a crude mixture of the resin-free peptide intermediate, and the crude mixture is contacted with the basic deblock solution in step (b). In some embodiments, Lixisenatide comprises the amino acid sequence of SEQ ID NO:2. In some embodiments, Lixisenatide has the amino acid sequence of SEQ ID NO:2. In some embodiments, the composition is administered subcutaneously. In some embodiments, the composition is administered by subcutaneous injection, such as by using an injection pen. In some embodiments, the composition is administered once daily. In some embodiments, the composition is administered within an hour before any meal of the day. In some embodiments, the composition is administered before the same meal every day. In some embodiments, the composition is administered at a dose of 10 μg daily. In some embodiments, the composition is administered at a dose of 20 μg daily.

In some embodiments, there is provided a method of treating diabetes (such as type II diabetes) in an individual in need thereof, comprising administering to the individual an effective amount of a composition comprising a plurality of Lixisenatide, wherein the percentage of Lixisenatide having an N-terminal D-histidine in the composition is less than about 1% (such as less than about any of 0.75%, 0.5%, or 0.3%), and wherein the plurality of Lixisenatide is prepared by steps comprising: (a) optionally synthesizing a resin-bound peptide intermediate on a resin using Fmoc-protected amino acids according to the sequence of Lixisenatide; (b) contacting the resin-bound peptide intermediate with an acidic cleavage solution to provide a resin-free peptide intermediate, wherein the N-terminus of the resin-bound peptide intermediate is protected by an Fmoc group; (c) contacting the resin-free peptide intermediate with a basic deblock solution to remove the Fmoc group from the N-terminus of the resin-free peptide intermediate to provide a reaction mixture comprising Lixisenatide; (d) optionally contacting the reaction mixture with an acidic neutralization solution; and (e) optionally purifying Lixisenatide (such as using chromatography) from the reaction mixture to provide the plurality of Lixisenatide. In some embodiments, the acidic cleavage solution comprises TFA (such as a solution comprising TFA, TIS, EDT, and H₂O, for example, at a volume ratio of about 94:2:2:2). In some embodiments, the deblock solution comprises piperidine (such as about 15% to about 25% (v/v) piperidine in ACN and water). In some embodiments, step (b) provides a crude mixture of the resin-free peptide intermediate, and the crude mixture is contacted with the basic deblock solution in step (c). In some embodiments, the acidic neutralization solution comprises TFA (such as 50% TFA in water). In some embodiments, Lixisenatide comprises the amino acid sequence of SEQ ID NO:2. In some embodiments, Lixisenatide has the amino acid sequence of SEQ ID NO:2. In some embodiments, the composition is administered subcutaneously. In some embodiments, the composition is administered by subcutaneous injection, such as by using an injection pen. In some embodiments, the composition is administered once daily. In some embodiments, the composition is administered within an hour before any meal of the day. In some embodiments, the composition is administered before the same meal every day. In some embodiments, the composition is administered at a dose of 10 μg daily. In some embodiments, the composition is administered at a dose of 20 μg daily.

The effective doses of the composition may be determined by the attending clinician and may be further dependent upon the efficacy of the particular peptides in the composition, as well as upon the age, weight and condition of the individual. The optimal mode of administration of the composition to an individual depends on factors known in the art, such as the particular disease or disorder, the desired effects, and the type of patient. In some embodiments, the composition is administered to a human individual. In some embodiments, the composition is administered to a vertebrate, such as other primates, farm animals such as swine, cattle and poultry, and sports animals and pets such as horses, dogs and cats.

The compositions (such as Exenatide compositions, Lixisenatide compositions, or Liraglutide compositions) of the present application of the invention may be administered in any suitable form that will provide sufficient levels of the compositions for the intended purpose. Intravenous administration is a useful route of administration, although other parenteral routes can also be employed, where parenteral as used herein includes subcutaneous injections, intravenous injection, intraarterial injection, intramuscular injection, intrasternal injection, intraperitoneal injection, or infusion techniques. The compositions can also be administered orally or enterally, which is a preferred route when compatible with the absorption of the composition. Where the pharmacokinetics of the compositions are suitable, the compositions can also be administered sublingually, by buccal administration, subcutaneously, by spinal administration, by epidural administration, by administration to cerebral ventricles, by inhalation (e.g. as mists or sprays), rectally, or topically in unit dosage formulations containing conventional nontoxic pharmaceutically acceptable carriers, excipients, adjuvants, and vehicles as desired. The compositions may be administered directly to a specific or affected organ or tissue. In some embodiments, the composition is administered parentally. In some embodiments, the composition is administered subcutaneously. In some embodiments, the composition is administered by injection. In some embodiments, the composition is administered by peripheral injection, such as injection to the skin of the abdomen, upper leg, or upper arm. The compositions are mixed with pharmaceutically acceptable carriers, excipients, adjuvants, and vehicles appropriate for the desired route of administration.

Suitable dosages for administering the compositions (such as Exenatide compositions, Lixisenatide compositions, or Liraglutide compositions) include, but are not limited to, at least about any of 0.1 μg, 1 μg, 2 μg, 5 μg, 10 μg, 15 μg, 20 μg, 25 μg, 30 μg, 40 μg, 50 μg, 100 μg, 250 μg, 500 μg, 1 mg, 5 mg, 10 mg, 20 mg, 50 mg, 100 mg, or more. In some embodiments, the composition is administered at a dose of any of about 0.1 μg to about 100 mg, about 1 μg to about 5 μg, about 5 μg to about 10 μg, about 10 μg to about 20 μg, about 20 μg to about 100 μg, about 100 μg to about 200 μg, about 100 μg to about 1 mg, about 1 mg to about 10 mg, about 1 μg to about 30 μg, about 1 μg to about 50 μg, about 30 μg to about 50 μg, about 1 μg to about 500 μg, or about 10 mg to about 100 mg. In some embodiments, the composition is administered at a dose of any of about 0.001 μg/kg to about 1 mg/kg, about 0.005 μg/kg to about 0.2 μg/kg, about 0.02 μg/kg to about 0.1 μg/kg, about 0.05 μg/kg to about 0.1 μg/kg once, or about 0.1 μg/kg to about 0.5 μg/kg per dose. In some embodiments, for a patient with diabetes who weighs in the range from about 70 kilograms (average for the type I diabetic) to about 90 kilograms (average for the type II diabetic), for example, the composition may be administered at a dose of about 10 μg to about 120 μg per day in single or divided doses (such as twice or three times) by injection. Doses of the Exenatide, Lixisenatide, or Liraglutide composition may be lower if given by continuous infusion. Doses of the Exenatide, Lixisenatide, or Liraglutide composition may be higher if given by non-injection methods, such as oral, buccal, sublingual, nasal, pulmonary or skin patch delivery. For example, oral dosages may be from about 500 to about 12,000 μg per day in single or divided doses, such as from about 500 to about 5,000 μg per day. Pulmonary dosages may be about 100 to about 12,000 μg per day in single or divided doses, such as about 500 to 1000 μg per day. Nasal, buccal and sublingual dosages may be about 100 to about 12,000 μg per day in single or divided doses.

The effective amount of the composition may be administered in a single dose or in multiple doses. Exemplary dosing frequencies include, but are not limited to, daily, daily without break, weekly, weekly without break, weekly for two out of three weeks, weekly for three out of four weeks, once every three weeks, once every two weeks, monthly, every six months, yearly, etc. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the composition is administered once daily, twice daily, three times daily, or four times daily. In some embodiments, there is no break in the dosing schedule.

The administration of the composition can be extended over an extended period of time, such as from 1 day to about a week, from about a week to about a month, from about a month to about a year, from about a year to about several years. In some embodiments, the composition is administered over a period of at least any of about 1 day, 2 days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

The composition may be administered singly or in combination with another therapeutic agent, for example, a glucose-lowering agent, a gastric emptying modulating agent, a lipid lowering agent, or a food intake inhibitor agent. Therapeutically effective amounts of the Exenatide, Lixisenatide, or Liraglutide composition for use in the control of blood glucose or in the control of gastric emptying and in conditions in which gastric emptying is beneficially slowed or regulated are those that decrease post-prandial blood glucose levels, preferably to no more than about 8 or 9 mM or such that blood glucose levels are reduced as desired. In diabetic or glucose intolerant individuals, plasma glucose levels are higher than in normal individuals. In such individuals, beneficial reduction or “smoothing” of post-prandial blood glucose levels may be obtained. As will be recognized by those in the field, an effective amount of therapeutic agent will vary with many factors including the patient's physical condition, the blood sugar level or level of inhibition of gastric emptying to be obtained, or the desired level of food intake reduction, and other factors.

The present application also provides a composition comprising a plurality of resin-free peptide intermediates, wherein each resin-free peptide intermediate comprises an Fmoc-protected N-terminal histidine and unprotected amino acid side chains. In some embodiments, the resin-free peptide intermediates comprises the amino acid sequence of SEQ ID NO:1. In some embodiments, the resin-free peptide intermediates comprises the amino acid sequence of SEQ ID NO:2. In some embodiments, the resin-free peptide intermediates comprises the amino acid sequence of SEQ ID NO:3. In some embodiments, the resin-free peptide intermediate comprises less than about 1% (such as less than about any of 0.75%, 0.5% or 0.3%) resin-free peptide intermediates having an incorrect N-terminal histidine enantiomer (such as D-histidine). The compositions are useful for synthesizing a peptide having an N-terminal histidine, or a composition comprising a plurality of the peptides thereof.

### IV. Kits, and Articles of Manufacture

The present invention further provides kits, and articles of manufacture (such as commercial batches) comprising any of the compositions described herein.

The kits of the invention are in suitable packaging. Suitable packaging include, but is not limited to, vials, cans (such as pressurized cans), bottles, jars, flexible packaging (e.g., Mylar or plastic bags), cartridges, syringes, and the like. The present application thus also provides articles of manufacture, which include vials, cans (such as pressurized cans), bottles, jars, flexible packaging, cartridges, syringes, injection pens, and the like.

In some embodiments, the kit comprises an injection device, such as a prefilled syringe or disposable pen. In some embodiments, the kit comprises the composition in a lyophilized form, for example, contained in a vial. Kits may optionally provide additional components such as buffers (e.g., reconstitution solvent) and interpretative information.

In some embodiments, the kit comprises instructions. The instructions may contain information generally related to the administration of the compositions, such as the effective amount, frequency, and administration routes. The instructions may further contain information related to the storage, reconstitution, and/or safety information for administering the compositions.

The kits and articles of manufacture may contain unit doses, bulk packages (e.g., multi-dose packages) or sub-unit doses. For example, kits may be provided that contain a sufficient amount of the composition for any of 1, 2, 3, 4, 5, 6, 7, 8, 9, 10, 20, 50, 100 or more administrations.

In some embodiments, there is provided a commercial batch of any of the compositions (such as pharmaceutical compositions) described herein. “Commercial batch” used herein refers to a batch size that is at least about 1 gram (by amount of the peptides having the N-terminal histidine). In some embodiments, the batch size is at least about any of 1, 2, 5, 10, 30, 40, 50, 60, 70, 80, 90, 100, 150, 200, 250, 300, 350, 400, 450, 500, 550, 600, 650, 700, 750, 800, 850, 900, 1000, 1500, 2000, 2500, 3000, 3500, 4000, 4500, 5000, or 10000 grams (by amount of the peptides having the N-terminal histidine). In some embodiments, the commercial batch comprises a plurality of vials comprising any of the compositions (such as pharmaceutical compositions) described herein. In some embodiments, the commercial batch comprises at least about any of 100, 150, 200, 100, 150, 200, 250, 300, 350, 400, 450, 500, 550, 600, 650, 700, 750, 800, 850, 900, 1000, 1500, 2000, 2500, 3000, 3500, 4000, 4500, 5000, 10000, 12000, 14000, 16000, 18000, 20000, 22000, 24000, 26000, 28000, 30000, 32000, 34000, 36000, 38000, 40000, 42000, 44000, 46000, 48000, or 50000 vials. For example, each vial contains about any of 100, 200, 300, 400, 500, 600, 700, 800, 900, or 1000 mg of the composition (such as pharmaceutical composition). In some embodiments, the commercial batch comprises at least about any of 100, 150, 200, 100, 150, 200, 250, 300, 350, 400, 450, 500, 550, 600, 650, 700, 750, 800, 850, 900, 1000, 1500, 2000, 2500, 3000, 3500, 4000, 4500, 5000, 10000, 12000, 14000, 16000, 18000, 20000, 22000, 24000, 26000, 28000, 30000, 32000, 34000, 36000, 38000, 40000, 42000, 44000, 46000, 48000, or 50000 injection pens or syringes containing the composition (such as pharmaceutical composition). For example, each injection pen or syringe contains at least about any of 100 μg, 120 μg, 150 μg, 200 μg, 300 μg, 400 μg, 500 μg, 600 μg, 700 μg, 800 μg, 900 μg, 1 mg, 2 mg, 5 mg, 10 mg, or more of the composition (such as pharmaceutical composition). In some embodiments, the pharmaceutical composition in the commercial batch is a liquid suspension. In some embodiments, the pharmaceutical composition in the commercial batch is a lyophilized powder.

### Exemplary Embodiments

The present application provides the following embodiments:

1. A method of preparing a peptide having an N-terminal histidine, comprising:

(a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; and
(b) contacting the resin-free peptide intermediate with a basic deblock solution to remove the Fmoc group from the N-terminus of the resin-free peptide intermediate to provide the peptide having the N-terminal histidine.

2. The method of embodiment 1, further comprising synthesizing the resin-bound peptide intermediate on a resin using Fmoc-protected amino acids according to the sequence of the peptide having the N-terminal histidine.

3. The method of embodiment 1 or embodiment 2, wherein the side chain of the N-terminal histidine of the resin-bound peptide intermediate is protected by a group selected from trityl (Trt), 4-methyltrityl (Mtt), and p-methoxytrityl (Mmt).

4. The method of any one of embodiments 1-3, wherein the acidic cleavage solution comprises trifluoroacetic acid (TFA).

5. The method of any one of embodiments 1-4, wherein the basic deblock solution comprises piperidine.

6. The method of any one of embodiments 1-5, wherein the basic deblock solution comprises acetonitrile (ACN).

7. The method of any one of embodiments 1-6, wherein the basic deblock solution comprises water.

8. The method of any one of embodiments 5-7, wherein the concentration of the piperidine in the deblock solution is about 15% to about 25% by volume.

9. The method of any one of embodiments 5-8, wherein the resin-free peptide intermediate is contacted with the basic deblock solution for about 15 minutes to about 30 minutes.

10. The method of any one of embodiments 1-9, wherein step (a) provides a crude mixture of the resin-free peptide intermediate, and the crude mixture is contacted with the basic deblock solution in step (b).

11. The method of any one of embodiments 1-10, further comprising contacting the reaction mixture comprising the peptide having the N-terminal histidine with an acidic neutralization solution after step (b).

12. The method of any one of embodiments 1-11, further comprising purifying the peptide having the N-terminal histidine.

13. The method of embodiment 12, wherein the peptide having the N-terminal histidine is purified using a C18 column.

14. The method of any one of embodiments 1-13, wherein the N-terminal histidine is an L-histidine.

15. The method of any one of embodiments 1-14, wherein the peptide is Exenatide.

16. The method of embodiment 15, wherein the peptide has the amino acid sequence of SEQ ID NO:1.

17. The method of any one of embodiments 1-14, wherein the peptide is Lixisenatide.

18. The method of embodiment 17, wherein the peptide has the amino acid sequence of SEQ ID NO:2.

19. A composition comprising a plurality of peptides having an N-terminal histidine prepared by the method of any one of embodiments 1-18, wherein the percentage of peptides having an N-terminal D-histidine in the composition is less than about 1%.

20. A method of treating a disease or condition in an individual in need thereof, comprising administering to the individual an effective amount of the composition of embodiment 19.

21. The method of embodiment 20, wherein the disease is type II diabetes.

22. A commercial batch of the composition of embodiment 19.

23. The commercial batch of embodiment 22, wherein the size of the commercial batch is about 1 gram to about 10 Kg.

24. A composition comprising a plurality of resin-free peptide intermediates, wherein each resin-free peptide intermediate comprises an Fmoc-protected N-terminal histidine and unprotected amino acid side chains.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further details of the invention are illustrated by the following non-limiting Examples. The disclosures of all references in the specification are expressly incorporated herein by reference.

### EXAMPLES

The examples below are intended to be purely exemplary of the invention and should therefore not be considered to limit the invention in any way. The following examples and detailed description are offered by way of illustration and not by way of limitation.

### Example 1—SCX HPLC Method for Determining D-his Impurity in Exenatide

In this example, SCX HPLC was used to determine the level of D-His impurity in Exenatide or other peptides having an N-terminal L-His. One advantage of the SCX HPLC method is preservation of the integrity of the peptide sample. Thus, the SCX HPLC method can be used as an in-process control for Exenatide synthesis and purification.

50 mg D-His-Exenatide and 100 mg Exenatide were mixed together in deionized (DI) water and lyophilized into a powder. The powder was then dissolved in buffer to prepare a 1 mg/mL sample solution for analysis. A HPLC equipped with an SCX LUNA® column (Phenomenex) was used to analyze the sample solution. The sample solution was eluted along a concentration gradient, and buffers used for the mobile phase were MPA (0.05 M KH₂PO₄ in ACN and water at 1:1 v/v ratio) and MPB (0.05 M KH₂PO₄, 0.1 M NaClO₄ in ACN and water at 1:1 v/v ratio).

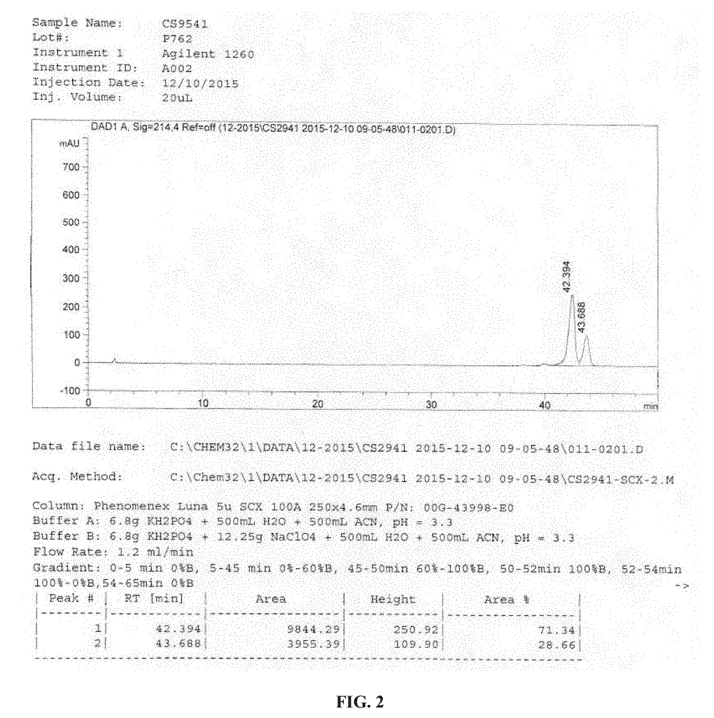

FIG. 2 shows the chromatography of the sample solution. The peaks for Exenatide (42.39 min) and D-His-Exenatide (43.68 min) were well resolved. The profile of the SCX HPLC separation can be used to determine the content of D-His-Exenatide impurity in any batch of Exenatide.

### Example 2—Optimization of Exenatide SPPS

To investigate the cause of the high level of D-His-Exenatide impurity in the synthesized Exenatide batches, we first synthesized a peptide resin using an Fmoc-Rink amide MBHA resin (sub 0.3-0.4), and deprotected the N-terminal Fmoc group to obtain the deFmoc peptide resin Gly²-38-Ser³⁹-NH₂ (SEQ ID NO:4). Full Exenatide peptide was synthesized from the Gly²-38-Ser³⁹-NH₂ peptide resin by coupling Fmoc-His(Trt)-OH to the Gly²-38-Ser³⁹-NH₂ peptide resin, deprotecting the N-terminal Fmoc group and amino acid side chains, cleaving the peptide from the resin, and C18 HPLC purification. The synthesis followed the exact procedure in the GMP manufacturing process for Exenatide synthesis.

The D-His impurity in the resulting Exenatide was determined using amino acid analysis and GC/MS methods. We found that the D-His-Exenatide was present in several lots of Exenatide Acetate at a level of 1.8%-2.9%. We also further found that the second highest D-amino acid impurity in Exenatide was D-Ile (<0.5%), including <0.1% D-isoleucine, <0.2% L-allo-isoleucine, and <0.2% D-allo-isoleucine. The D-amino acid impurities from the rest of the amino acid residues in Exenatide were all below 0.2%.

SEQ ID No: 4
(Gly²-38-Ser39-NH₂)
Gly-Glu-Gly-Thr-Phe-Thr-Ser-Asp-Leu-Ser-Lys-Gln-
Met-Glu-Glu-Glu-Ala-Val-Arg-Leu-Phe-Ile-Glu-Trp-
Leu-Lys-Asn-Gly-Gly-Pro-Ser-Ser-Gly-Ala-Pro-Pro-
Pro-Ser-NH₂

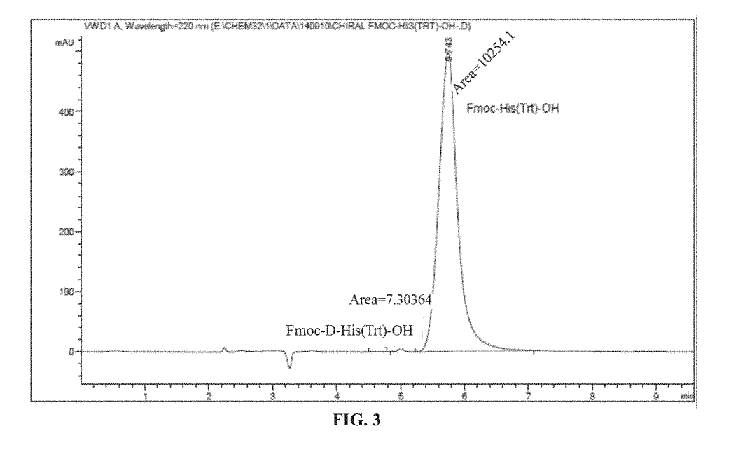

The enantiomeric purity of Fmoc-His(Trt)-OH was determined by SCX HPLC. The content of Fmoc-D-His(Trt)-OH was 0.07%. FIG. 3 shows the chromatography of the Fmoc-His(Trt)-OH monomer. Because Fmoc-His(Trt)-OH is the only raw material to introduce D-His, the content of L-His was >99.9%, and the content of D-His (as Fmoc-D-His(Trt)-OH) was <0.1% in the starting material, which was below the level of the D-His impurity in the final product.

Thus, D-His could be introduced to Exenatide from solid phase synthesis, resin cleavage, and/or purification process. Both resin cleavage and purification steps do not involve chiral carbon related reactions, thus SPPS is likely to be the cause of D-His impurity. It is known that base-catalyzed and acid-catalyzed epimerization of amino acids may occur during a peptide-bond formation reaction. Additionally, racemization can take place via 5(4H)-oxazolone formation. Based on the GC/MS results, our current coupling methods did not in general lead to racemization of the amino acid moieties, except for Fmoc-His(Trt)-OH.

His (including Fmoc-His(Trt)-OH) racemization tends to take place not only during coupling reactions, but also under basic conditions. Deprotection of the Fmoc group from His (i.e., deFmoc reaction) with 20% Pip in DMF takes place in basic conditions, which can increase the likelihood of His racemization.

To optimize the SPPS condition, numerous trials were done to minimize the D-His formation, including low coupling temperature, short coupling time, short deFmoc time, and low deFmoc temperature. So far, we could only decrease the D-His impurity to 1.5-1.8%.

Since His is the last amino acid to be coupled in Exenatide synthesis, we hypothesize that a final on-resin deFmoc step could be altered in the SPPS process. Therefore, the following two alternative synthesis processes were tested.

### Use of Boc-His(Boc)-OH

Boc-His(Boc)-OH could be used instead of Fmoc-His(Trt)-OH for the last coupling in Exenatide synthesis. The Boc protection group as side chain protecting group could withdraw the free n-nitrogen of the imidazole moiety, which contributes to the racemization.

After the last Boc-His(Boc)-OH was attached to the Gly²-38-Ser39-NH₂ peptide resin, the peptide was cleaved off the resin and purified before the D-His impurity was determined by SCX HPLC. The cleavage condition followed the exact procedure in the manufacturing process for Exenatide cleavage. However, the purification process was simplified in one step using a gradient of 0.1% TFA and ACN over a C18 column.

Table 1 below lists the coupling conditions and the testing results for D-His formation from the solid phase synthesis of the last coupling. Boc-His(Boc)-OH yielded a lower content of D-His Exenatide impurity.

TABLE 1
Exenatide synthesis using Boc-His(Boc)-OH.

| | | | | | | D-His |
|---|---|---|---|---|---|---|
| | | | | | | determination |
| | Protected AA | | | Ninhydrin | | after |
| Sample | for the last | Peptide | | test for the | Purification | purification via |
| ID | coupling | resin | Coupling condition | completion | Condition | SCX HPLC |
| 3 | Boc-His(Boc)- | Gly²-38- | Resin/AA/DIC/HOBt, 1/8/8/8 eq | Negative, | 0.1% TFA(MPA); | 0.484% (D-His) |
| | OH | Ser-NH₂ | at 15° C., 4.5 hrs | complete | 100% ACN(MPB) | 0.24% (D-His, |
| | | | | | | GC/MS) |
| 12 | Boc-His(Boc)- | Gly²-38- | Resin/AA/DIC/Cl-HOBt, 1/5/5/5 | Negative, | 0.1% TFA(MPA); | 0.625%; 0.654% |
| | OH | Ser-NH₂ | eq at rt, 15 hrs | complete | 100% ACN(MPB) | (D-His) |

Other building blocks for the last His coupling were also investigated, including Boc-His(Trt)-Gly-OH, Boc-His(Trt)-OH, and Boc-His(Boc)-OH, and Boc-His(Boc)-OH yielded the lowest level of D-His impurity. The coupling condition was Resin/AA/DIC/HOBt (1/8/8/8 eq) at 15° C., 4.5 hrs. The lowest D-His impurity level achieved was <0.3% by GC/MS (<0.7% by SCX HPLC) after purification.

### Final Fmoc Deprotection in Solution

Although racemization is observed during the last His coupling to the peptide resin using building blocks, such as Fmoc-His(Trt)-OH and Boc-His(Boc)-OH, further racemization takes place under basic conditions leading to the D-His-Exenatide impurity, when the final on-resin deFmoc step was performed using piperidine. Therefore, the final deFmoc step was optimized in order to minimize the D-His impurity level before purification.

Without being bound by any theory or hypothesis, the racemization of L-His during the deFmoc reaction can be accelerated by the conjugated π electron system of the imidazole side chain, which is further enriched by the Trt protecting group, under basic conditions. To minimize the racemization reaction, the Trt group could be removed before performing the deFmoc step on the N-terminal His. A peptide with the N-terminal Fmoc group can be first cleaved off the resin with TFA after full length-peptide resin synthesis. The TFA cleavage also cleaves off all side chain protection groups from the peptide. Fmoc de-protection can be performed subsequently in a mixture with ACN and water.

In particular, after coupling of Fmoc-His(Trt)-OH (last AA) to the Gly²-38-Ser39-NH₂ peptide resin, Exenatide peptide resin was washed and dried. TFA cleavage (TFA/TIS/H₂O/EDT) was performed to provide Fmoc-Exenatide, which had all side chain protection groups removed by the process. Fmoc-Exenatide crude treatment performed with C18 column in 0.01-0.05% TFA buffer system yielded >90% of Fmoc-Exenatide product. The peptide fractions were combined, and piperidine (10-20% in volume) was added to remove the Fmoc group in solution over the course of 10-30 min to yield Exenatide crude peptide. After filtration through a 1 micron filter, the crude peptide was loaded onto the C18 column for purification. SCX HPLC was used to determine the content of D-His Exenatide in the crude peptide, and after purification. Results from small-scale synthesis demonstrated that D-His racemization could be controlled below 1% before the C18 purification process, which meets the <1% D-form impurity criteria according to USP.

The above optimized methods can be used to synthesize other peptides with an N-terminal His, such as Lixisenatide and Liraglutide, to obtain peptides with low D-His impurities.

### Example 3—Large-Scale Synthesis of Exenatide

First, solid phase peptide synthesis was carried out to provide the resin-bound peptide intermediate. A Rink amide (RAM) resin having a 4′-{(R,S)-alpha-{1-(9-Fluorenyl)methoxycarbonylamino}-2,4-dimethoxybenzyl}-phenoxyacetic acid linker, was used. Fmoc-amino acids were sequentially coupled to the resin using a threefold molar excess of the respective amino acid in the sequence. Briefly, Fmoc-amino acids (3 eq.) with 1-Hydroxybenzotriazole (HOBt) (3 eq.) were dissolved in DMF/DCM, and DIC (3 eq.) was added to activate the Fmoc-amino acids. While DIC was combined with the amino acid/HOBt solution, the whole mixture was transferred into the reaction vessel with drained resin in 5 minutes to start the coupling. Coupling reaction was allowed for at least 2 hours, and Kaiser Test was used to assess the progress of the reaction. For difficult coupling reactions as determined by the Kaiser Test, longer coupling time was allowed. After coupling, reaction reagents were filtered off, and the resin was washed with DMF three times, followed by Fmoc deprotection (i.e. deFmoc) using 20% Pip in DMF. Fmoc deprotection was carried out twice, for 5-10 min and 20-30 min respectively with 20% Pip in DMF, and 6-8 washing steps were followed with alternating DMF and DCM washes. After deprotection of the Fmoc, the next amino acid was attached following the same procedure. The coupling cycle was repeated with the respective building blocks according to the target sequence until the second to the last amino acid was coupled, which was Gly². No Fmoc deprotection step was carried out after the coupling of the Fmoc-His(Trt)-OH for the last amino acid (i.e., the N-terminal amino acid) His¹. A resin-bound peptide intermediate was obtained after the last coupling step.

To carry out the acidic cleavage step, dry resin-bound peptide intermediate (100 g) was weighed and transferred to a new reaction vessel. TFA solution containing appropriate scavengers (1 L) was added. The reaction vessel was stoppered and left to stand at room temperature with stirring to avoid aggregation. The resin beads were dispersed throughout the TFA solution (TFA/TIS/EDT/H2O, 94/2/2/2 (v/v)). After 4 hours of reaction, the resin was removed by filtration under pressure and washed twice with TFA. The filtrates were combined to obtain the resin-free Fmoc-peptide.

To carry out the deblock step, about 3 g-4 g of the resin-free Fmoc-peptide was dissolved in 80 mL of ACN/water solution (1/1 in volume), and 20 mL of piperidine was added to the solution. Optionally, the resin-free Fmoc-peptide was purified using a C18 column, and the combined fractions were directly used in the deblock step. The reaction mixture was stirred for 20 min at room temperature.

After 20 minutes of the deblock reaction, 40 mL of pre-mixed ice water with 50% TFA was poured into the reaction solution with stirring to neutralize the piperidine. The pH of the mixture was checked to ensure the solution pH was below 4. The solution was then filtered through a 0.45-1 micron filter. The filtrate was directly loaded onto a C18 column and purified using a concentration gradient set up by Buffer A (0.1% TFA water solution), and Buffer B (100% ACN). Fractions with >97% peptide were combined, and the content of D-His impurity was determined. Exenatide produced using the large-scale manufacture process described herein yielded about 0.6-1.2% peptides having D-His impurity as determined by SCX HPLC. The USP standard method for quantifying D-His content in Exenatide is GC/MS, and it is known that levels measured using GC/MS are about 0.7 times the levels measured using SCX HPLC. Thus, the percentage of D-His-containing peptides in Exenatide produced using the large-scale manufacture process described herein would be about 0.42% to about 0.84% when determined by GC/MS, which is below the 1% criterion according to USP.

What is claimed is:

1. A method of preparing a peptide having an N-terminal histidine, comprising:
(a) contacting a resin-bound peptide intermediate having the N-terminal histidine with an acidic cleavage solution to provide a resin-free peptide intermediate, wherein the N-terminus of the resin-bound peptide intermediate is protected by an Fmoc group; and
(b) contacting the resin-free peptide intermediate with a basic deblock solution to remove the Fmoc group from the N-terminus of the resin-free peptide intermediate to provide the peptide having the N-terminal histidine.

2. The method of claim 1, further comprising synthesizing the resin-bound peptide intermediate on a resin using Fmoc-protected amino acids according to the sequence of the peptide having the N-terminal histidine.

3. The method of claim 1, wherein the side chain of the N-terminal histidine of the resin-bound peptide intermediate is protected by a group selected from trityl (Trt), 4-methyltrityl (Mtt), and p-methoxytrityl (Mmt).

4. The method of claim 1, wherein the acidic cleavage solution comprises trifluoroacetic acid (TFA).

5. The method of claim 1, wherein the basic deblock solution comprises piperidine.

6. The method of claim 5, wherein the concentration of the piperidine in the deblock solution is about 15% to about 25% by volume.

7. The method of claim 5, wherein the resin-free peptide intermediate is contacted with the basic deblock solution for about 15 minutes to about 30 minutes.

8. The method of claim 1, wherein step (a) provides a crude mixture of the resin-free peptide intermediate, and the crude mixture is contacted with the basic deblock solution in step (b).

9. The method of claim 1, further comprising contacting the reaction mixture comprising the peptide having the N-terminal histidine with an acidic neutralization solution after step (b).

10. The method of claim 1, further comprising purifying the peptide having the N-terminal histidine.

11. The method of claim 1, wherein the N-terminal histidine is an L-histidine.

12. The method of claim 1, wherein the peptide is Exenatide.

13. The method of claim 12, wherein the peptide has the amino acid sequence of SEQ ID NO:1.

14. The method of claim 1, wherein the peptide is Lixisenatide.

15. The method of claim 14, wherein the peptide has the amino acid sequence of SEQ ID NO:2.

16. A composition comprising a plurality of peptides having an N-terminal histidine prepared by the methods of claim 1, wherein the percentage of peptides having an N-terminal D-histidine in the composition is less than about 1%.

17. A method of treating a disease or condition in an individual in need thereof, comprising administering to the individual an effective amount of the composition of claim 16.

18. A commercial batch of the composition of claim 16.

19. The commercial batch of claim 18, wherein the size of the commercial batch is about 1 gram to about 10 Kg.

20. A composition comprising a plurality of resin-free peptide intermediates, wherein each resin-free peptide intermediate comprises an Fmoc-protected N-terminal histidine and unprotected amino acid side chains.
